(19) United States
(12) Patent Application Publication
(10) Pub. No.: US 2002/0136779 A1
(43) Pub. Date: Sep. 26, 2002

(54) Pharmaceutical compound preparation comprising a parathyroid hormone preparation and a calcium/phosphate preparation

(75) Inventor: Paul Lehmann, Worms (DE)

Correspondence Address: BRUCE LONDA, NORRIS, MCLAUGHLIN & MARCUS, P.A., 220 EAST 42ND STREET, 30TH FLOOR, NEW YORK, NY, 10017, US

(73) Assignee: SCIL Diagnostics GmbH, Martinsried (DE)

(21) Appl. No.: 10/102,238

(22) Filed: Mar. 20, 2002

Related U.S. Application Data
(62) Division of application No. 09/446,420, filed on Feb. 28, 2000.
Application No. 09/446,420 is a 371 of international application No. PCT/EP98/03554, filed on Jun. 12, 1998.

(30) Foreign Application Priority Data
Jun. 19, 1997 (DE) 197 110 026.8

(51) Int. Cl. A61K 38/29; A61K 33/42; A61K 31/7024

(52) U.S. Cl. 424/602; 514/23; 514/2

(57) ABSTRACT

The present invention relates to pharmaceutical combination preparations comprising a) single administration forms of a parathyroid hormone preparation (PTH) suitable for single dosage of the active substance in a maximum amount of 20 μg of PTH, and b) a calcium/phosphate preparation wherein the calcium and phosphate compounds may be present in separate administration forms or in an integrated administration form.

The present invention relates to pharmaceutical combination preparations including a parathyroid hormone preparation (PTH) suitable for single dosage of the active substance with a maximum quantity of 20 μg PTH, and a calcium/-phosphate preparation, wherein the calcium and phosphate compounds may be present in separate administration forms or in an integrated administration form, methods of preparing same, and their use. In particular, the combination preparations are used in the treatment of bone metabolic diseases.

Parathyroid hormone (PTH), a hormone (84 amino acids) of the parathyroid glands, is an important regulator in maintaining the calcium level in the body. PTH is capable of stimulating the bone formation or bone resorption where it acts as a regulatory hormone on a number of enzymes, including ornithine decarboxylase and adenylate cyclase (cAMP synthesis), among others. In the event of calcium deficiency, PTH mobilizes calcium from the bones, reduces calcium excretion of the kidneys and at the same time, improves absorption of calcium from the intestine by increased synthesis of 1,25-dihydroxycholecalciferol. Owing to the effect on these target organs, a normalization of the calcium level is achieved. Conversely, in the event of an elevated calcium level, the incorporation of calcium in bones is stimulated. In addition, PTH exhibits a mitogenic effect, particularly a stimulation of osteoblasts and chondrocytes.

The osteogenous activity of PTH has been observed in animal experimental studies on rats as well as in clinical studies on osteoporotic patients, and has been described in the technical literature (Selye, Endocrinology 16 (1932), 547-558; Hefti et al., Clin. Sci. 62 (1982), 389-396; Gunnes-Hey et al., Metab. Bone Dis. Relat. Res. 5 (1984), 177-181; Reeve et al., Br. Med. J. 280 (1980), 1340-1344; Slovik et al., J. Bone Miner. Res. 1 (1986), 377-381, EP 0,197,514).

Bone tissue is the storage organ for calcium ions, from which they can be mobilized under deficient conditions. Frequently, bone tissue diseases are processes occurring predominantly in association with diseases of other organs or the entire metabolism. By interfering with the hormonal regulation of mineralization and bone formation and bone resorption, they may give rise to demineralization phenomena (osteomalacia), bone resorption (osteoporosis) or bone deformation.

Histologically, bone tissue consists of 5-10 μm thick lamellas of bone substance and cells incorporated therein, the osteocytes, which are arranged around the small vessels of the bone extending along the longitudinal axis. The osteoblasts, which are situated on the surface of the bone, are involved in the structure of the bone substance. Their activity is in a hormonally controlled equilibrium with bone resorption by the osteoclasts.

The bone mineral required for bone stability, which makes up 70% of the bone substance, consists of calcium and phosphate. The organic proportion, which makes up 30% of the bone substance, is predominantly composed of a proteoglycan matrix and a fibrous network of type 1 collagen.

In native bone, the mineral substance consists of an amorphous calcium phosphate phase and a crystalline apatite, part of which contains carbonate and is characterized by disorders, preferably hydroxylapatite. Apatite has a large interior surface where ion exchange processes are possible, e.g. exchange of hydroxyl ions for fluoride or Co₃²⁻ ions to form fluorapatite and carbonate apatite, respectively.

In particular, the stem cells which synthesize apatite and its most important storage protein, i.e., collagen, thereby consuming calcium and phosphorus, are controlled by PTH which regulates the calcium balance, the stem cells being developed to form osteoblasts.

The osteoclasts, which are responsible for bone resorption, are cell formations which have a high phagocytosis performance (macrophages) and are formed from the bone marrow stem cells. They are produced by granulocytopoiesis, they belong to the reticulo-endothelial system, make up about 5-8% of the leukocyte number, and settle in bones. In bone metabolism, hydroxylapatite is “labilized” by the osteoclasts, i.e., the apatite is degraded down to a basic skeleton. If disorders are absent, this basic substance is deposited by the osteoblasts in the intercellular space of bones and used for further bone synthesis.

As has been explained, the transport of calcium and phosphate from the bones to the developing osteoblasts takes place under the influence of PTH. Conversely, calcium and phosphate are transported to the bones after the apatite has been synthesized. PTH, which functions to differentiate as many stem cells into osteoblasts as possible in order to produce apatite in an amount as high as possible, therefore consumes calcium and phosphate. As a consequence, the calcium and phosphate levels decrease, thereby causing a loss of apatite in the bones.

Accordingly, the bone metabolism may involve the following critical items:

a) insufficient differentiation of stem cells by PTH,

b) “Labilization” of hydroxylapatite by the osteoclasts,

c) insufficient transport of calcium and phosphate to the osteoblasts.

U.S. Pat. No. 4,833,125 describes the use of PTH or active fragments thereof in combination with vitamin D or a dietary calcium supplement in a kit to increase the bone matter. As a result of such a combined use, a synergistic effect of PTH has been observed.

As can be inferred from the specification, the hPTH fragment 1-34 at an extremely high dosage of 400-500 U/day=400-500 μg/day and a dietary calcium supplement are preferably used, wherein the calcium amount exceeds the level of normal calcium food intake, and an extremely high average value of 2000 mg calcium/day is mentioned as upper limit.

However, such high PTH and calcium concentrations result in various drawbacks. Thus, such high PTH concentrations may give rise to hyperparathyroidism. As can be seen in Table 2 of U.S. Pat. No. 4,833,125, the urinary calcium values show a massive increase during PTH-calcium therapy. Moreover, high PTH concentrations result in an increased absorption of calcium from the intestine, so that lesions of the kidneys may occur. Also, a high PTH concentration inactivates EPO, thereby reducing the erythropoiesis. Simultaneously, the high calcium supply under such extreme conditions of therapy may cause disorders in iron absorption (Gunshin et al., Nature 388, 482-488, 1997). Thus, the above document fails to disclose a practicable therapy regimen for optimum adjustment and treatment of bone metabolic diseases.

It has now been found that the combination preparations described below allow a surprisingly advantageous treatment of bone metabolic diseases. Therefore, the invention is directed to a combination preparation comprising a parathyroid hormone preparation (PTH) including a maximum quantity of 20 μg of PTH, which is suitable for single dosing of the active substance, and, in addition, a calcium/phosphate preparation, wherein the calcium and phosphate compounds may be present in separate administration forms or in an integrated administration form. According to the invention, the calcium/phosphate preparation may also comprise a single compound containing both calcium and phosphorus. This combination preparation is suitable in the therapy of bone metabolic diseases.

In addition to a PTH preparation, the combination preparation in a preferred embodiment includes a preparation having a calcium phosphate complex compound. In a particularly preferred fashion, it contains an apatite compound of formula Ca²⁺[Ca₃ (PO₄)₂]₃²⁻ which preferably can be present in the form of a gluconate. However, the combination preparation may also include a calcium preparation, e.g. calcium gluconate, and a phosphate preparation, e.g. glucose-1-phosphate or potassium hydrogen phosphate.

The preferred ratio of calcium and phosphate (or phosphorus) in the combination preparation is from 11:5 to 9:7, preferably 10:6.

In a preferred variation, the combination preparation includes a PTH preparation having 0.5-20 μg of PTH, and a calcium/phosphate preparation, preferably apatite, having a total amount of calcium and phosphate of 280-320 mg, preferably 300 mg. Another variation comprises preparations containing 0.5-20 μg of PTH, 20-300 mg of calcium, and 10-150 mg of phosphorus (or 30-450 mg of phosphate). These values are to be interpreted in such a way that one or more appropriate calcium/phosphate preparations are to be applied in an amount so as to supply 20-300 mg of calcium and 10-150 mg of phosphorus (or 30-450 mg of phosphate).

In the meaning of the present invention, the term “PTH” is understood to include those peptides as well which are derived from the natural PTH peptide of 84 amino acids by deletions, substitutions or variations of one or more amino acids. In particular, peptide fragments truncated at the C-terminus are possible, such as PTH(1-34), PTH(1-35), PTH(1-36), or PTH(1-37). These PTH derivatives have been described in EP 0,497,915, WO 93/15109, EP 0,301,484, or WO 90/10067. According to the invention, naturally occurring PTH, chemically synthesized PTH, or PTH produced by genetic engineering can be used, particularly human PTH (hPTH). PTH fragments having the same effect may also be used, and these can be produced by cleaving naturally occurring PTH and by chemical-synthetic or genetic engineering methods (DE 37 25 319; Sömjen et al., Biochem. J. 272, 781-5 (1990)).

The combination preparations of the present invention may include the PTH preparation and the calcium/phosphate preparation formulated together in a ready-for-sale packaging unit (so-called combination package). In addition, the drug packages may either contain a suitable amount of PTH preparation or a suitable amount of a calcium/phosphate preparation in the form of a single preparation, the single preparations with respect to the amount of ingredients being formulated in a way so as to allow combined administration with the respective other preparation in the meaning of the invention. In these cases, the manufacturer or drug importer generally encloses an instruction leaflet with the preparations which is prescribed by law in many countries and includes instructions or information on the combined administration of the single preparations. Similarly, this applies for drug packagings including a suitable amount of PTH and a suitable amount of a calcium and a phosphate compound.

In the meaning of the invention, oral or parenteral administration forms are possible as calcium/phosphate preparations. In principle, these forms can be single preparations containing a physiologically tolerable calcium salt and a phosphate compound or a calcium phosphate complex compound as active substance, or combination preparations which, in addition to said physiologically tolerable preparations, include other active substances such as vitamins, folic acid, thiamine chloride, riboflavin, pyridoxine, ascorbic acid, nicotinamide, etc.

The PTH preparation and calcium/phosphate preparation can be administered in the form of separate pharmaceutical formulations (free combination) simultaneously or successively as well. Such a free combination, which may be provided in a single packaging unit, offers the advantage of high flexibility.

As a rule, the free combination is provided in the form of a single packaging unit comprising at least two receptacles, the first one being a suitable administration form of PTH (lyophilized product, injection or infusion solution), and the second one representing a suitable administration form of the calcium phosphate or calcium and phosphate preparation. In this way, each patient may individually be provided with a directly assignable quantity of PTH and calcium/phosphate compound. In addition, these combination preparations offer the advantage of more success in therapy because an optimally adjusted quantity of single preparations is determined each time, and confusion with other commercially available single preparations offered in varying dosages is largely excluded.

Moreover, the combination preparations according to the invention minimize the risk of an accidentally excessive calcium and phosphate administration which possibly may occur when applying conventional calcium and phosphate preparations from separate drug packages together with PTH. The combination preparations of the invention ensure safe therapy and easy handling. In the present case it is also possible to employ one active substance as an injection solution and the other active substance as an administration form for oral application.

In those cases where PTH is provided as a lyophilized product, the drug packages (combination packages) will include an appropriate amount of PTH in glass ampoules or in carpules. The calcium/phosphate preparation may be provided in a solid form (tablets, powders, granulates, lyophilized products, etc.) or in a liquid form in separate receptacles. In addition, the combination package includes a reconstituting solution to dissolve either the PTH lyophilized product alone or together with the solid calcium/phosphate preparation. In case the calcium/phosphate preparation is provided as a ready-made solution, said solution can be mixed with the PTH solution if joint application is intended. In principle, the calcium/phosphate preparation may also be provided as a concentrate to be added to conventional infusion solutions, thereby permitting a more gradual application over several hours.

Another possibility in the meaning of the invention is to provide the respective single preparations of PTH and calcium and phosphate compounds as independent drugs, the single preparations being formulated in a way so as to include the required amounts of single substances for the combination of PTH and calcium and phosphate according to the invention. Such single preparations may also include appropriate written informational statements, e.g. in the form of instruction leaflets or package imprintings referring to the combined administration together with the respective other single preparation in an amount as required. Such information is also relevant in context with approvals according to drug law for trading such preparations, or serves as technical information in the drug market.

When using the combination preparations, PTH and calcium and phosphate compounds may also be administered in a so-called fixed combination, i.e., in a single pharmaceutical formulation wherein both compounds are included. For example, this can be an injection solution or an infusion solution or a lyophilized product thereof filled in ampoules, for example. The fixed combination of the respective active substances in the form of a lyophilized product offers the advantage of easy and safe handling. By adding pharmaceutically conventional injection media, the lyophilized product is dissolved in the ampoule and applied intravenously.

A preferred packaging unit in the meaning of the invention comprises a PTH preparation having a maximum of 20 μg of PTH and a preparation including calcium and phosphate compounds in a single administration form or in separate administration forms, the calcium/phosphorus ratio being from 11:5 to 9:7.

In a particularly preferred fashion, it comprises a preparation having 0.5-20 μg of PTH and a calcium/phosphate preparation having a total amount of calcium and phosphate 300-500 mg, and/or a calcium preparation having 20-300 mg of calcium and a phosphate preparation having 10-150 mg of phosphorus in separate administration forms as injection or infusion solutions, or as lyophilized products, or in an integrated administration form.

The pharmaceutical administration forms are produced according to conventional methods known in the galenic art, using conventional pharmaceutical adjuvants.

When performing the combined therapy using the combination preparation of the invention, the apatite and collagen filling state of the bones has to be determined using various diagnostic parameters. The following were found to be particularly relevant and informative:

a) determination of the calcium x phosphate product in serum,

b) determination of the osteocalcin matrix protein in serum,

c) determination of collagen via its markers.

The target values normally indicating a well-adjusted bone metabolism can be inferred from Table 1:

TABLE 1

| | | | Product Ca × PO₄ | |
|---|---|---|---|---|
| | | | in serum | Urine |
| Analyte | Serum | | [mg/dl]² | [mmol/l] |
| Ca | 2.5 | 10 | 40 | <5 |
| Phosphate | 1.3 | 4 | | <20 |

| | |
|---|---|
| AP, bone | <150 U/l |

| | |
|---|---|
| Osteocalcin | 5-100 μg/l |
| Crosslinks | |
| Pyrodinoline | <100 μmol/mol creatinine |
| 3-Hydroxypyrodinoline | <20 μmol/mol creatinine |
| NTx (= N-terminal | Depending on method |
| crosslinked peptide | |
| PTH | 10-70 ng/l |

Essentially, the following diagnostic parameters must therefore be monitored when performing the therapy:

a) Ca×PO₄ in serum in (mg/dl)², as well as Ca and phosphate in urine;

b) apatite filling level in collagen via detection of bone ALP, pyrodinoline (“crosslinks”), 3-hydroxyproline, and NTx;

c) concentration of osteocalcin which is also capable of apatite storage and transport.

For diagnostic investigations, the product of calcium×phosphate represents the most important quantity and is determined in the serum. The bone was found to be filled sufficiently with Ca and PO₄ when the product of Ca×PO₄ was 30-50 (mg/dl)² where the target value should be 40 (mg/dl)² (J. E. van Nuwenborg et al., Euro. J. Clin. Biochem. 1997, 335(4), 297-300). A value of >60 (mg/dl)² Ca×PO₄ must be regarded as an indication for organ calcification. At values of <25 (mg/dl)² there is a risk of hypocalcemia and hypophosphatemia.

The normal values for calcium in serum range from 2.2 to 2.6 mmol/l which corresponds to 8.6-10.2 mg/dl (calcium). The normal values for phosphate range from 1.0 to 1.5 mmol/l which corresponds to 2.7-4.5 mg/dl (phosphate), resulting in a product of Ca×PO₄ which may be regarded as acceptable at values between 23 (mg/dl)² (8.6×2.7) and 46 (mg/dl)² (10.2×4.5).

Referring to the composition of the hydroxylapatite occurring in bone, an optimum loading capacity is present at a product of Ca×PO₄ of about 40 (mg/dl)² (cf., Table 2).

TABLE 2

| | mmol/l | | mmol/l |
|---|---|---|---|
| Caₛₑᵣᵤₘ | 2.2-2.6 | Phosphateₛₑᵣᵤₘ | 1.0-1.5 |
| Caₐₚₐₜᵢₜₑ | 2.2-2.6 | Phosphateₐₚₐₜᵢₜₑ | 1.3-1.6 |

Apatite: Ca₁₀(PO₄)₆(OH)₂

Apatite: Ca₁₀(PO₄)₆(OH)₂

The detection of collagen, which is the most important storage protein of apatite, is performed indirectly via markers of bone formation in the serum, such as bone ALP and terminal propeptides eliminated during formation of collagen fibrils, and via markers of bone resorption in urine, such as pyrodinoline (crosslinks), 3-hydroxyproline or NTx (N-terminal crosslinked peptide).

The matrix protein osteocalcin, enabled by its glutamic acid residues to bind to crystals of the calcium mineral hydroxylapatite incorporated in bones, also occurs in serum at a concentration of about 5-100 μg/l (normal value; depending on method).

When performing the combined therapy, the combination preparation of the invention therefore permits an easy decision as to the maximum weekly dosage.

For successful therapy in osteoporosis patients, preferably using rhPTH and an adequate apatite supply in order to achieve a target value of 40 (mg/dl)² of serum Ca×PO₄, the following therapy regimen preferably is recommended, wherein the risk of apatite overload is avoided: In a correction phase, e.g. in latent apatite deficiency (30 (mg/dl)² of Ca×PO₄ or even smaller values), 7×400 mg of calcium and phosphate in the form of apatite (in addition to Ca and phosphate in food) are administered per week and simultaneously, 7×5-20 μg of rhPTH 3 times a week. As a rule, the target value of 40 (mg/dl)² serum Ca×PO₄ is reached after about 20-40 weeks.

To maintain this value, additional administration of a combination preparation containing 5-10 μg of PTH and a total amount of calcium and phosphate compounds of about 300 mg is recommended, the ratio of calcium/phosphorus ranging from 11:5 to 9:7. When applying 5-10 μg of rhPTH three times a week and once per week later on, a dosage of 300 mg of apatite daily is recommendable, where the target value of 40 (mg/dl)² of Ca×PO₄ in the serum should be monitored. In case the Ca×PO₄ value should drop again, administration of 5-10 μg of rhPTH three times a week is repeated.

At the beginning of rhPTH therapy and about 3 weeks after the end of the correction phase, the entire bone parameters, hematological parameters, iron parameters, and thyroid gland parameters should be determined. It is recommendable to check these parameters twice a year.

The parameters and frequencies of determination can be inferred from Table 3.

TABLE 3
Apatite substitution in osteoporosis patients under rhPTH therapy

| | Target | |
|---|---|---|
| Diagnostic parameters | values | Frequency of determination |
| Bone parameters | | |
| Creatinine clearance | | |
| Ca in urine | <20 mg/dl | |
| PO₄ in urine | <80 mg/dl | |
| K, Mg in urine | | Every month during correction |
| | | phase |
| Ca in serum | 10 mg/dl | Quarterly during maintenance |
| | | phase |
| PO₄ in serum | 4 mg/dl | |
| Ca × PO₄ in serum | 40 | |
| | (mg/dl)² | |
| K, Mg in serum | | |
| 25-(OH)-D₃, serum | | |
| PTH, serum | | Quarterly |
| AP, bone, serum | | |
| Osteocalcin, serum | | |
| “Cross labs”, urine | | Every six months |
| Hydroxyproline, urine | | |
| Bone density | | Annually |
| Hematological parameters | | |
| Hemoglobin | 10-12 g/dl | Every month during correction |
| Hematocrit | 30-36% | and maintenance phases |
| Reticulocytes | 10-15% | |
| Folate | | Every six months during correc- |
| Vitamin B12 | | tion and maintenance phases |
| Iron parameters | | |
| Ferritin (F) | 100-400 | At beginning of correction phase |
| | ng/ml | and 3 weeks after the end of |
| Transferrin saturation | 20-35% | correction phase, quarterly |
| Hypochromic erythrocytes | <10% | during maintenance phase |
| Thyroid gland parameters | | |
| TSH, basic | | Every six months |
| FT3 | | |
| FT4 | | |

Therefore, the present invention relates to pharmaceutical combination preparations comprising a maximum of 20 μg of parathyroid hormone (PTH) and calcium and phosphate compounds, wherein the compounds may be present in separate administration forms or in an integrated administration form. In particular, combination preparations including PTH and a calcium phosphate complex compound are possible as preferred embodiments. Combination preparations containing an apatite compound of formula Ca²⁺[Ca₃(PO₄)₂]₃²⁻ as calcium phosphate complex compound, preferably in the form of a gluconate, are particularly preferred. Other preferred combination preparations are those including PTH, a calcium and a phosphate compound, particularly those including calcium gluconate as calcium compound, or glucose-1-phosphate or potassium hydrogen phosphate as phosphate compound. Other preferred combination preparations are those wherein the ratio of calcium/phosphate (phosphorus) is from 11:5 to 9:7, preferably 10:6, and those containing 0.5-20 μg of PTH, 20-300 mg of calcium and 10-150 mg of phosphorus (or 30-450 mg of phosphate), or a total amount of calcium and phosphate of 300-500 mg, preferably 400 mg. The invention is also directed to pharmaceutical packaging units comprising a PTH preparation including 0.5-20 μg of PTH and a calcium/phosphate preparation including a total amount of calcium and phosphate of 300-500 mg (Ca+P), or a calcium preparation including 20-300 mg of calcium and a phosphate preparation including 10-150 mg of phosphorus (or 30-450 mg of phosphate) in separate administration forms or in an integrated administration form.

The present invention also relates to methods of producing pharmaceutical combination preparations as specified above, wherein PTH, calcium and phosphate compounds are formulated with pharmaceutically conventional vehicles or adjuvants and provided in an integrated or separate administration form. In addition, the invention relates to the use of PTH and calcium and phosphate compounds in the production of combination preparations for treating bone metabolic disorders.

Furthermore, the invention relates to methods of determining the hydroxylapatite filling level of bones in a sample of body fluids, wherein the product of calcium×phosphate and the concentrations of osteocalcin and collagen are determined, particularly in the determination of bone metabolic disorders.

The invention will be illustrated in more detail with reference to the following example.

### EXAMPLE

Patients having manifest bone metabolic disorders, whose osteocalcin value is below 8 μl/l and whose calcium×phosphate value is below 20, are treated three times a week using 20 μg of hPTH. In addition, the patients receive 300 mg of calcium and 150 mg of phosphorus three times a week, preferably in the form of a Ca[Ca₃(PO₄)₂]₃ gluconate solution which is infused. This treatment is continued until the osteocalcin value and the calcium x phosphate value are in the normal range (duration of treatment about twenty weeks).

1. A pharmaceutical combination preparation, comprising
a) single administration forms of a parathyroid hormone preparation (PTH), suitable for single dosage of the active substance in a maximum amount of 20 μg of PTH, and
b) a calcium/phosphate preparation wherein the calcium and phosphate compounds are present in separate administration forms or in an integrated administration form.

2. The combination preparation according to claim 1, characterized in that the calcium/phosphate preparation includes a calcium phosphate complex compound.

3. The combination preparation according to claim 2, characterized in that the calcium phosphate complex compound is an apatite compound of formula Ca^{2 +}[Ca₃(PO₄)₂]₃²⁻; preferably in the form of a gluconate.

4. The combination preparation according to claim 1, characterized in that the calcium/phosphate preparation contains calcium gluconate.

5. The combination preparation according to claim 1, characterized in that the calcium/phosphate preparation contains glucose-i-phosphate or potassium hydrogen phosphate.

6. The combination preparation according to any of claims 1 to 5, characterized in that the ratio of calcium/phosphorus is from 11:5 to 9:7, preferably 10:6.

7. The combination preparation according to any of claims 1 to 6, characterized in that said preparation includes a PTH preparation having 0.5-20 μg of PTH, and a calcium/phosphate preparation having a total amount of calcium and phosphate compounds of 300-500 mg, preferably 400 mg, the ratio of calcium/phosphorus ranging from 11:5 to 9:7.

8. The combination preparation according to any of claims 1 to 7, characterized in that said preparation includes a calcium preparation having 20-300 mg of calcium, and a phosphate preparation having 10-150 mg of phosphorus.

9. A method of producing the combination preparations according to claims 1 to 8, characterized in that a maximum of 20 μg of PTH of a PTH preparation in the form of single administration forms, 20-300 mg of calcium and 10-150 mg of phosphorus of a calcium/phosphate preparation or a total amount of calcium and phosphate compounds of 300-500 mg (Ca+P) having a ratio of calcium/phosphorus ranging from 11:5 to 9:7 are formulated either together or separately with pharmaceutically conventional vehicles or adjuvants, and the respective preparations are provided in the form of combination preparations.

10. Use of PTH preparations in the production of combination preparations including a maximum of 20 μg of PTH in the form of single administration forms for combined administration together with calcium/phosphate preparations, the calcium and phosphate compounds being present in separate administration forms or in an integrated administration form.

11. A pharmaceutical packaging unit, comprising a PTH preparation having a maximum of 20 μg of PTH, and a calcium/phosphate preparation, wherein the PTH preparation and the calcium/phosphate preparation may be present in an integrated administration form or in separate administration forms and also, the calcium/phosphate preparation may be present in the form of a calcium and a phosphate preparation in separate administration forms or in an integrated administration form.

12. The pharmaceutical packaging unit according to claim 11, characterized in that the PTH preparation includes 0.5-20 μg of PTH, the calcium preparation includes 20-300 mg of calcium, and the phosphate preparation includes 10-150 mg of phosphorus, and that the preparations are present in separate administration forms or in an integrated administration form as injection or infusion solutions or as lyophilized products.

13. The pharmaceutical packaging unit according to claim 11, characterized in that the PTH preparation includes 0.5-20 μg of PTH, and the calcium/phosphate preparation includes a total amount of 300-500 mg of calcium and phosphorus at a ratio of calcium/phosphorus ranging from 11:5 to 9:7, and that the preparations are present in separate administration forms or in an integrated administration form as injection or infusion solutions or as lyophilized products.

14. A method of determining the hydroxylapatite filling level of bones in a sample of body fluids, wherein the product of calcium×phosphate and the concentrations of osteocalcin and collagen are determined.
